SEQUENCE LISTING (134 sequences)

SEQ ID NO: 1 - DNA, 16 bp, artificial sequence
  Probe
acggcgtcgccctgaa

SEQ ID NO: 2 - DNA, 16 bp, artificial sequence
  Probe
ggggcgttccgctcaa

SEQ ID NO: 3 - DNA, 20 bp, artificial sequence
  Probe
acttatccccacgaaagtcg

SEQ ID NO: 4 - DNA, 19 bp, artificial sequence
  Probe
tttccatcctcgtcacccc

SEQ ID NO: 5 - DNA, 26 bp, artificial sequence
  Probe
tctgttcaacaccattaattttcact

SEQ ID NO: 6 - DNA, 24 bp, artificial sequence
  Probe
cacaaacatcctttattaatcccg

SEQ ID NO: 7 - DNA, 23 bp, artificial sequence
  Probe
cacaaagagtcttcattaagccg

SEQ ID NO: 8 - DNA, 21 bp, artificial sequence
  Probe
agcaatgcacctctggtcaat

SEQ ID NO: 9 - DNA, 25 bp, artificial sequence
  Probe
agcaaagaaacaatggtcaattact

SEQ ID NO: 10 - DNA, 25 bp, artificial sequence
  Probe
agttacacacactgatcaacagtgg

SEQ ID NO: 11 - DNA, 22 bp, artificial sequence
  Probe
gcaacgcaaaccagatcaatag

SEQ ID NO: 12 - DNA, 25 bp, artificial sequence
  Probe
cccatagtaaccaacacctaagaca

SEQ ID NO: 13 - DNA, 24 bp, artificial sequence
  Probe
acaaaaccaagttcaatagaaggg

SEQ ID NO: 14 - DNA, 19 bp, artificial sequence
  Probe
agcatgatcaaaacagagg

SEQ ID NO: 15 - DNA, 20 bp, artificial sequence
  Probe
ctgctacgaggcaaccttgg

SEQ ID NO: 16 - DNA, 19 bp, artificial sequence
  Probe
cgctcgatggctaacctga

SEQ ID NO: 17 - DNA, 21 bp, artificial sequence
  Probe
gcagacaaggagtggtggaca

SEQ ID NO: 18 - DNA, 20 bp, artificial sequence
  Probe
aagacacgaccagcagcacc

SEQ ID NO: 19 - DNA, 27 bp, artificial sequence
  Probe
aaaacacgtctagtagtaccatcacaa

SEQ ID NO: 20 - DNA, 16 bp, artificial sequence
  Probe
acacgaccggccgctc

SEQ ID NO: 21 - DNA, 22 bp, artificial sequence
  Probe
gaggaggaaatgaaggctcaat

SEQ ID NO: 22 - DNA, 17 bp, artificial sequence
  Probe
tggagctccgctgtggc

SEQ ID NO: 23 - DNA, 22 bp, artificial sequence
  Probe
agcagctcttacgctacatgga

SEQ ID NO: 24 - DNA, 22 bp, artificial sequence
  Probe
cgtatgttgtcaaggttgagcc

SEQ ID NO: 25 - DNA, 21 bp, artificial sequence
  Probe
ggacccatgaaattggtgatg

SEQ ID NO: 26 - DNA, 24 bp, artificial sequence
  Probe
gctgtgggagtaatgaatcaagtt

SEQ ID NO: 27 - DNA, 22 bp, artificial sequence
  Probe
aatgtggaagttgggaatgtca

SEQ ID NO: 28 - DNA, 22 bp, artificial sequence
  Probe
ggggatgtcaaattttagctcc

SEQ ID NO: 29 - DNA, 20 bp, artificial sequence
  Probe
tggggaaatctggactggag

SEQ ID NO: 30 - DNA, 20 bp, artificial sequence
  Probe
tggggagctctggactggag

SEQ ID NO: 31 - DNA, 20 bp, artificial sequence
  Probe
agggatggaaaggatcaccg

SEQ ID NO: 32 - DNA, 20 bp, artificial sequence
  Probe
gctccacgcttgcttgctta

SEQ ID NO: 33 - DNA, 22 bp, artificial sequence
  Probe
acggtcaacaggacacatcaat

SEQ ID NO: 34 - DNA, 24 bp, artificial sequence
  Probe
tgatgaatgactcaatggctaaga

SEQ ID NO: 35 - DNA, 20 bp, artificial sequence
  Probe
agtttgttcgtggaccctcg

SEQ ID NO: 36 - DNA, 24 bp, artificial sequence
  Probe
aaaggatgccgagtctataaaagc

SEQ ID NO: 37 - DNA, 19 bp, artificial sequence
  Probe
ttttcgtggcactttggca

SEQ ID NO: 38 - DNA, 22 bp, artificial sequence
  Probe
gtgccgaaagaattggttgaat

SEQ ID NO: 39 - DNA, 20 bp, artificial sequence
  Probe
tgcagtctccacctcagcaa

SEQ ID NO: 40 - DNA, 23 bp, artificial sequence
  Probe
tttgccaggaatgactacaaaag

SEQ ID NO: 41 - DNA, 17 bp, artificial sequence
  Probe
cggacctgcggtgggta

SEQ ID NO: 42 - DNA, 26 bp, artificial sequence
  Probe
tgtggagctatgttggaaactatatg

SEQ ID NO: 43 - DNA, 22 bp, artificial sequence
  Probe
ctggaactgcctttgatactgg

SEQ ID NO: 44 - DNA, 21 bp, artificial sequence
  Probe
ccggaactgcctttgatactg

SEQ ID NO: 45 - DNA, 25 bp, artificial sequence
  Probe
ctgtggagctatgttggaaactatg

SEQ ID NO: 46 - DNA, 22 bp, artificial sequence
  Probe
tgggaactgcatctgatactgg

SEQ ID NO: 47 - DNA, 28 bp, artificial sequence
  Probe
ctgtggaactatgttggaaactatatgt

SEQ ID NO: 48 - DNA, 24 bp, artificial sequence
  Probe
ccatagtaggctttggaaactgtt

SEQ ID NO: 49 - DNA, 22 bp, artificial sequence
  Probe
aggaattgcatttcagactggg

SEQ ID NO: 50 - DNA, 19 bp, artificial sequence
  Probe
cgcagcctgcacttgaaac

SEQ ID NO: 51 - DNA, 20 bp, artificial sequence
  Probe
ggggagggtcattggaaact

SEQ ID NO: 52 - DNA, 25 bp, artificial sequence
  Probe
attgtaggctttggaaactgtttaa

SEQ ID NO: 53 - DNA, 16 bp, artificial sequence
  Probe
gagcgtgcgggcgata

SEQ ID NO: 54 - DNA, 27 bp, artificial sequence
  Probe
cagttgtatgcattggaaactattaat

SEQ ID NO: 55 - DNA, 20 bp, artificial sequence
  Probe
cgggaactgcgttctgaact

SEQ ID NO: 56 - DNA, 21 bp, artificial sequence
  Probe
gtggagggtcattggaaactg

SEQ ID NO: 57 - DNA, 24 bp, artificial sequence
  Probe
ccatagtaggctttggaaactgtt

SEQ ID NO: 58 - DNA, 21 bp, artificial sequence
  Probe
caaattacccaatcccgacac

SEQ ID NO: 59 - DNA, 21 bp, artificial sequence
  Probe
ccgtgcttcttagagggactg

SEQ ID NO: 60 - DNA, 24 bp, artificial sequence
  Probe
tttgtcagcttcttaaagggactt

SEQ ID NO: 61 - DNA, 23 bp, artificial sequence
  Probe
cacttcttagagggactttgcgt

SEQ ID NO: 62 - DNA, 16 bp, artificial sequence
  Primer
tccaagccccgcaagc

SEQ ID NO: 63 - DNA, 23 bp, artificial sequence
  Primer
ctatgcaacattcgtcgagtttc

SEQ ID NO: 64 - DNA, 21 bp, artificial sequence
  Primer
tcgggacatgccaagtaaagt

SEQ ID NO: 65 - DNA, 20 bp, artificial sequence
  Primer
ggattttcgttgttccagcc

SEQ ID NO: 66 - DNA, 20 bp, artificial sequence
  Primer
ccgtagcccataatggaacg

SEQ ID NO: 67 - DNA, 17 bp, artificial sequence
  Primer
ggggtggtggatgtgcg

SEQ ID NO: 68 - DNA, 21 bp, artificial sequence
  Primer
tcaacactatggccgagcttt

SEQ ID NO: 69 - DNA, 21 bp, artificial sequence
  Primer
atgattgcggcgttaggtgac

SEQ ID NO: 70 - DNA, 24 bp, artificial sequence
  Primer
gataaaacatacgcggttcagagt

SEQ ID NO: 71 - DNA, 21 bp, artificial sequence
  Primer
ctcgcggtataatgcatgatg

SEQ ID NO: 72 - DNA, 22 bp, artificial sequence
  Primer
taaaacagcctgtgggttgttc

SEQ ID NO: 73 - DNA, 22 bp, artificial sequence
  Primer
taaaacagcctgtgggttgtac

SEQ ID NO: 74 - DNA, 19 bp, artificial sequence
  Primer
ggattagccgcattctggg

SEQ ID NO: 75 - DNA, 20 bp, artificial sequence
  Primer
aaaaccctttcccagccttg

SEQ ID NO: 76 - DNA, 19 bp, artificial sequence
  Primer
agtggggaccaggcatagg

SEQ ID NO: 77 - DNA, 22 bp, artificial sequence
  Primer
aaaacagctctggggttgtacc

SEQ ID NO: 78 - DNA, 20 bp, artificial sequence
  Primer
tcaccggggaaacagaagtg

SEQ ID NO: 79 - DNA, 21 bp, artificial sequence
  Primer
gtgaagcaaaacccatccaaa

SEQ ID NO: 80 - DNA, 21 bp, artificial sequence
  Primer
caatctgcgctgcaaacatat

SEQ ID NO: 81 - DNA, 21 bp, artificial sequence
  Primer
tgtcacagtgatggcaccaga

SEQ ID NO: 82 - DNA, 22 bp, artificial sequence
  Primer
gcagacaaatgttgggtcactc

SEQ ID NO: 83 - DNA, 18 bp, artificial sequence
  Primer
ttccccagaaccgaccct

SEQ ID NO: 84 - DNA, 23 bp, artificial sequence
  Primer
cagattccaatttcatgaccatg

SEQ ID NO: 85 - DNA, 20 bp, artificial sequence
  Primer
atgggtggatttggttctcg

SEQ ID NO: 86 - DNA, 20 bp, artificial sequence
  Primer
tgtcaatgcttcctttgcca

SEQ ID NO: 87 - DNA, 25 bp, artificial sequence
  Primer
agggagttcgtaaacagatatttgc

SEQ ID NO: 88 - DNA, 24 bp, artificial sequence
  Primer
agggagttcgtaaacagtaccttg

SEQ ID NO: 89 - DNA, 23 bp, artificial sequence
  Primer
cttagtcacagcgcttttgatga

SEQ ID NO: 90 - DNA, 20 bp, artificial sequence
  Primer
ttacagcattagccccgacc

SEQ ID NO: 91 - DNA, 20 bp, artificial sequence
  Primer
acaaaagcttcccctggaaa

SEQ ID NO: 92 - DNA, 19 bp, artificial sequence
  Primer
catgacccttggagtgggg

SEQ ID NO: 93 - DNA, 23 bp, artificial sequence
  Primer
aatgtctcctttatggctgatgc

SEQ ID NO: 94 - DNA, 20 bp, artificial sequence
  Primer
atgaggaccattgggcctct

SEQ ID NO: 95 - DNA, 21 bp, artificial sequence
  Primer
tgatgacatccatgatgcagc

SEQ ID NO: 96 - DNA, 19 bp, artificial sequence
  Primer
gcacgccaaattgaccaac

SEQ ID NO: 97 - DNA, 20 bp, artificial sequence
  Primer
atcaccatagtcgccgagcc

SEQ ID NO: 98 - DNA, 19 bp, artificial sequence
  Primer
cgagaaaccgcgtgtcaac

SEQ ID NO: 99 - DNA, 26 bp, artificial sequence
  Primer
atggctagaaatcttaggaatgctat

SEQ ID NO: 100 - DNA, 23 bp, artificial sequence
  Primer
gatcaaggcttgagcaatcagtt

SEQ ID NO: 101 - DNA, 24 bp, artificial sequence
  Primer
tttgtaggggtaaggataccgtta

SEQ ID NO: 102 - DNA, 22 bp, artificial sequence
  Primer
aacatcaagcaccgcctaaaaa

SEQ ID NO: 103 - DNA, 21 bp, artificial sequence
  Primer
ggagttcacggatcttcctcg

SEQ ID NO: 104 - DNA, 22 bp, artificial sequence
  Primer
ggtattctaaaccccgttcagc

SEQ ID NO: 105 - DNA, 21 bp, artificial sequence
  Primer
aaggagtcactgagccggact

SEQ ID NO: 106 - DNA, 24 bp, artificial sequence
  Primer
ctcatcataatcacgagcatcaaa

SEQ ID NO: 107 - DNA, 25 bp, artificial sequence
  Primer
tgatgctgaggtgtagacttgagac

SEQ ID NO: 108 - DNA, 26 bp, artificial sequence
  Primer
aacagtactagatgtgggagatgcat

SEQ ID NO: 109 - DNA, 26 bp, artificial sequence
  Primer
ttttgttctaaagggctctaagattt

SEQ ID NO: 110 - DNA, 23 bp, artificial sequence
  Primer
ggacatgggaggagctggtgggg

SEQ ID NO: 111 - DNA, 20 bp, artificial sequence
  Primer
accaggcggcgactaggaga

SEQ ID NO: 112 - DNA, 24 bp, artificial sequence
  Primer
tcccttatactggagatcctccat

SEQ ID NO: 113 - DNA, 20 bp, artificial sequence
  Primer
ccaggacacagtctgtttgt

SEQ ID NO: 114 - DNA, 21 bp, artificial sequence
  Primer
attgagggacgtgatgcagat

SEQ ID NO: 115 - DNA, 22 bp, artificial sequence
  Primer
caaagaagaaattggggatggt

SEQ ID NO: 116 - DNA, 23 bp, artificial sequence
  Primer
cgccactttgattttattcttcc

SEQ ID NO: 117 - DNA, 20 bp, artificial sequence
  Primer
ggcaggtccatgattggcac

SEQ ID NO: 118 - DNA, 20 bp, artificial sequence
  Primer
aactgaaaccaaggccctga

SEQ ID NO: 119 - DNA, 20 bp, artificial sequence
  Primer
cgcttggagaggatggatcc

SEQ ID NO: 120 - DNA, 25 bp, artificial sequence
  Primer
tgtgatcaaacttcctggtatcaga

SEQ ID NO: 121 - DNA, 21 bp, artificial sequence
  Primer
gggtggggctaggatgtaact

SEQ ID NO: 122 - DNA, 25 bp, artificial sequence
  Primer
caagcataacaatagattcaccctc

SEQ ID NO: 123 - DNA, 25 bp, artificial sequence
  Primer
caaacttgggctaattttggtaggt

SEQ ID NO: 124 - DNA, 22 bp, artificial sequence
  Primer
ggagattacagccccaacaaga

SEQ ID NO: 125 - DNA, 20 bp, artificial sequence
  Primer
ctgacccgtccccaactaca

SEQ ID NO: 126 - DNA, 26 bp, artificial sequence
  Primer
tttgaagaaggctatcatattcatgt

SEQ ID NO: 127 - DNA, 19 bp, artificial sequence
  Primer
cctgtgccgctagactccc

SEQ ID NO: 128 - DNA, 21 bp, artificial sequence
  Primer
tgagaaacggctaccacatcc

SEQ ID NO: 129 - DNA, 19 bp, artificial sequence
  Primer
ctgctggcaccagacttgc

SEQ ID NO: 130 - DNA, 16 bp, artificial sequence
  Primer
ccagcagccgcggtaa

SEQ ID NO: 131 - DNA, 18 bp, artificial sequence
  Primer
cgctcgttgcgggactta

SEQ ID NO: 132 - DNA, 22 bp, artificial sequence
  Primer
ggcttaatttgactcaacacgg

SEQ ID NO: 133 - DNA, 22 bp, artificial sequence
  Primer
ggcttaattcgactcaacacgg

SEQ ID NO: 134 - DNA, 15 bp, artificial sequence
  Primer
ggagcgacgggcggt

(19) United States
(12) Patent Application Publication
(10) Pub. No.: US 2005/0130186 A1
(43) Pub. Date: Jun. 16, 2005

(54) Meningitis detection chip and fabrication method thereof and method of detecting meningitis and primer set for meningitis detection

(76) Inventors: Kun-Shan Lu, Taipei (TW); Cheng-Yu Wei, Hualien City (TW); Yu-Jie Zhao, Shenyang City (CN)

Correspondence Address: J C PATENTS, INC., 4 VENTURE, SUITE 250, IRVINE, CA, 92618, US

(21) Appl. No.: 10/911,318

(22) Filed: Aug. 3, 2004

(30) Foreign Application Priority Data
Dec. 12, 2003 (TW) 92135134

(51) Int. Cl. C12Q 1/68; C12M 1/34; B05D 3/00

(52) U.S. Cl. 435/6; 435/287.2; 427/2.11

(57) ABSTRACT

A meningitis microarray detection chip includes a plurality of probes immobilized on a matrix, wherein each probe is selected from the group of deoxyribonucleotide sequences assigned to the SEQ ID NOs. 1 to 61. Since these probes are formed with deoxyribonucleotide sequences specific to meningitis, they can be used to detect meningitis.

### CROSS-REFERENCE TO RELATED APPLICATION

This application claims the priority benefit of Taiwan application serial no. 92135134, filed on Dec. 12, 2003.

### BACKGROUND OF THE INVENTION

1. Field of the Invention

The present invention relates to a detection device for an illness and a fabrication method thereof, a method for detecting the illness and a primer set for detecting the illness. More particularly, the present invention relates to a meningitis detection chip and a fabrication method thereof, a method for detecting meningitis and a primer set for meningitis detection.

2. Description of Related Art

Meningitis, encephalitis, meningoencephalitis are serious illnesses of the nervous system. A variety of pathogens is responsible for these neurological infections, and there are 10 most commonly seen pathogens. An inappropriate treatment of the disease can lead to a rapid development of a life-threatening infection. A prompt and accurate diagnosis is one vital factor for treating meningitis. However, the pathogenicity and the drug resistance are not the same for the different categories or the different types of pathogens. Therefore, clinical treatment of meningitis is very difficult. For example, symptoms of viral meningitis are similar to those of other types of meningitis. However, antibiotics are not effective for treating viral meningitis. Medical practitioner normally can not provide an immediate diagnosis on the type of meningitis, for example, whether it is bacterial or viral. As a result, the patient is first hospitalized and is given antibiotics intravenously. The specific treatment is only conducted after a bacterial culture on the cerebrospinal fluid is completed, which usually takes 48 to 72 hours. In the mean time, not only undue medical expenses are incurred the intervention to the illness is delayed. Although statistically, 90% of the cases are viral meningitis, the remaining cases, which are caused by other pathogens, would complicate the clinical diagnosis of the disease.

The traditional methods of examinations for cerebrospinal fluid include Gram's stain, antibody based immunology test and bacterial culture. Although the Gram's stain diagnostic test is simple and fast by using a microscope, a substantial amount of the bacteria is required. The antibody based immunology test is also rapid; however, it can only test one antibody at a time. Although bacterial culture is highly sensitive, this test requires a long time to produce meaningful results. Further, the application of antibiotics may lead to a pseudo negative result in the Grams' stain test or bacterial culture.

Microarray detection chip is one of the most important technological advancements in the high-tech field in recent years. Microarray detection chip is a high technology product that bases on a multidisciplinary effort from areas of physics, chemistry, microelectronics, precision machining and bioscience. Mircorarray detection chip can provide a large amount of probes with specific DNA sequences immobilized on a matrix, and a great deal of information is produced after the probe is reacted with a test sample (for example, DNA). Therefore, a microarray detection chip can be used for screening diseases. However, for different types of diseases, a great deal of effort must be devoted to the fabrication of the microarray detection chip in order to design a specific probe and a primer set to amplify the DNA fragment of the test sample of the patient. The current meningitis detection techniques still have not included a microarray detection chip for screening meningitis.

### SUMMARY OF THE INVENTION

Accordingly, the present invention provides a specific probe designed according to the various pathogens of meningitis and a corresponding primer set, and using this microarray detection chip technique to develop an accurate and rapid meningitis detection chip and a fabrication method thereof, and a detection method.

In accordance to the present invention, a fabrication method for a meningitis detection chip is provided, wherein the resulting detection chip can concurrently screen a plurality of meningitis pathogens.

The present invention also provides a microarray detection chip for meningitis, wherein problems of an extended waiting period for a test result and an inaccurate test result are resolved.

The present invention further provides a detection method for meningitis for accurately and rapidly detecting whether a patient has contracted meningitis and the type of meningitis.

The present invention further provides a primer set for meningitis detection for amplifying a specific deoxyribonucleic acid (DNA) fragment of a patient's sample.

The present invention provides a fabrication method for a meningitis detection chip, wherein the method includes designing a multiple of probe sequences and these probe sequences are formed with DNA sequences assigned to SEQ ID NOs. (Sequence Identifier Number) 1 to SEQ ID No. 61. The design of the probe sequences further includes a plurality of primer sets that corresponds to the probe sequences in order to amplify specific DNA fragments of the test sample from the patient. The primer sets comprises at least 38 primer sets that are formed with the DNA sequence assigned to the SEQ ID NOs. 62 to 134. A probe synthesis step is conducted to synthesize the probes formed with DNA sequences assigned to SEQ ID NOs. 1 to SEQ ID NO. 61. Thereafter, a spotting step is performed to spot respectively these probes on a matrix.

The present invention provides a microarray detection chip, wherein the detection chip is applicable for detecting whether a patient has contracted meningitis. This detection chip includes immobilizing a plurality of probes on a matrix, wherein each probe is selected from the group of the DNA sequences assigned to the SEQ ID NOs. 1-61.

The present invention provides a detection method for meningitis, wherein this method is applicable for detecting whether a patient has meningitis. This detection method includes providing the aforementioned microarray detection chip. After obtaining a DNA sample from the patient, a plurality primer sets is used to conduct a polymerase chain reaction (PCR) to amplify a specific DNA fragment and to obtain a PCR product. The primers sets used in the PCR are selected from the 38 primer sets, which are formed with the DNA sequences assigned to the SEQ ID NO. 62-134. A hybridization procedure is then conducted for this PCR product to react with the probes on the microarray detection chip. Thereafter, the test result from the microarray detection chip is analyzed.

Since the microarray detection chip of the present invention employs the DNA sequence specific to meningitis, this detection chip can be used to detect whether a patient has meningitis and the type of meningitis.

The present invention provides a primer set for detecting meningitis, wherein this primer set is selected from one of the 38 primer sets that are formed with the DNA sequences assigned to the SEQ ID NOs. 62-134.

Since these primer sets (SEQ ID NOs. 62-134) are related to the specific pathogens of meningitis, using these primer sets to amplify the specific DNA fragment of the patient and the detection method, whether the patient has meningitis and the type of meningitis can be confirmed.

It is to be understood that both the foregoing general description and the following detailed description are exemplary, and are intended to provide further explanation of the invention as claimed.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention.

FIG. 1 is a flow diagram illustrating the fabrication for a meningitis detection chip according to one embodiment of the invention.

FIG. 2 is a flow diagram illustrating the method of detecting meningitis according to one embodiment of the invention.

### DESCRIPTION OF THE EMBODIMENTS

The important aspects in the fabrication of a microarray detection chip are the designs of the probes and the primers set for amplifying the DNA segments of a patient's sample. The microarray detection chip can provide accurate information only with the design of specific probes and specific primer sets. The fabrication of the microarray detection chip for meningitis and the related detection method of the present invention are based on the aforementioned concepts. Although the disclosure herein refers to certain illustrated embodiments, it is to be understood that these embodiments are presented by way of example and not by way of limitation.

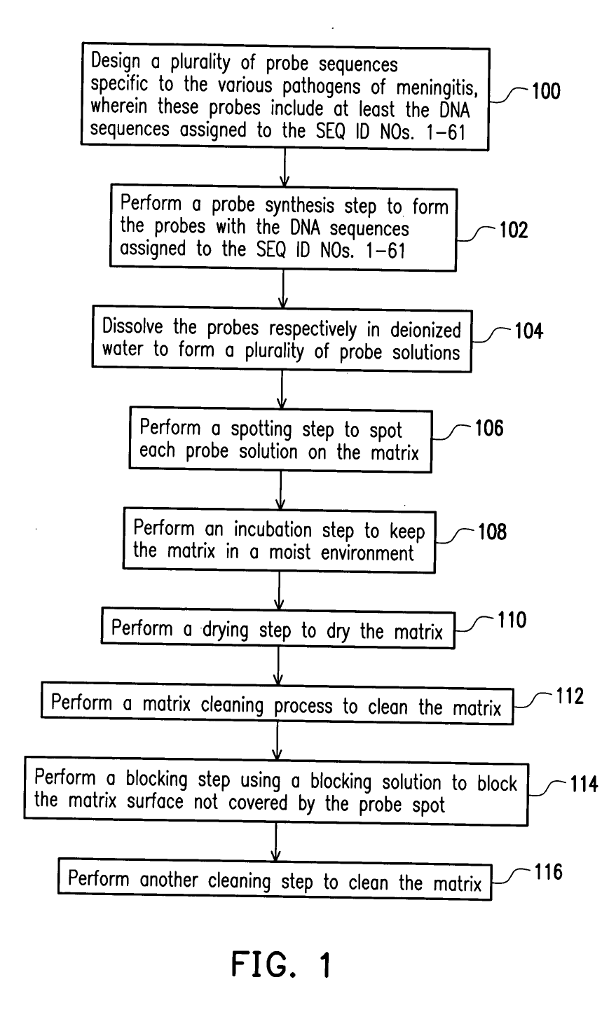

FIG. 1 is a flow diagram illustrating the fabrication method of a meningitis detection chip according to one embodiment of the invention.

Referring to FIG. 1, a plurality of probe sequences is designed specific to the various pathogens of meningitis, wherein these probe sequences include at least the deoxyribonucleotide sequences assigned to SEQ ID NOs. (Sequence Identification Number) 1-61 (step 100). These probe sequences, for example, are formed with 15 to 30 deoxyribonucleic acids. These probe sequences are designed according to the available gene sequences of the pathogens of meningitis obtained from a gene bank.

It is worth to note that the typical pathogens of meningitis include virus, bacteria, fungi and protozoa. Table 1 lists the pathogens of meningitis and the corresponding SEQ ID NOs. of the probe sequences.

TABLE 1

| | | SEQ ID NO. of the probe |
|---|---|---|
| Pathogens | | sequences |
| Virus | hhv1 | 1 |
| | hhv2 | 2 |
| | hhv3 | 3 |
| | hhv4 | 4 |
| | hhv5 | 5 |
| | hhv6 | 6 |
| | hhv7 | 7 |
| | coxsackievirus a9 | 8 |
| | coxsackievirus b4 | 9 |
| | coxsackievirus b5 | 10 |
| | enterovirus71 | 11 |
| | echovirus22 | 12 |
| | poliovirus | 13 |
| | la cross | 14 |
| | St. louis | 15 |
| | venezuelan equine | 16 |
| | west nile | 17 |
| | western equine (western) | 18 |
| | western equine (eastern) | 19 |
| | western equine (venezuelan) | 20 |
| | Japanese B | 21 |
| | central european | 22 |
| | russian | 23 |
| | Powassan | 24 |
| | Dengue | 25 |
| | Mumps | 26 |
| | Measles | 27 |
| | Adenovirus | 28 |
| | LCMV | 29 |
| | LCMV′ | 30 |
| | HIV-1 | 31 |
| | HIV-2 | 32 |
| | inluenza-a | 33 |
| | inluenza-b | 34 |
| | htlv-1 | 35 |
| | htlv-2 | 36 |
| | togavirus | 37 |
| | Rickettsia typhi | 38 |
| | Rabies virus | 39 |
| | Parvovirus B19 | 40 |
| Bacteria | Tropheryma whippelii | 41 |
| | Borrelia afzelii | 42 |
| | Bartonella henselae | 43 |
| | Brucella abortus \grave{ } Brucella | 44 |
| | canis \grave{ } Brucella melitensis \grave{ } | |
| | Brucella suis | |
| | Borrelia burgdorferi | 45 |
| | Escherichia coli | 46 |
| | Borrelia garinii | 47 |
| | Streptococcus pneumoniae R6 | 48 |
| | Haemophilus influenzae Rd | 49 |
| | Leptospira interrogans | 50 |
| | Bacillus subtilis | 51 |
| | Streptococcus milleri | 52 |
| | Mycobacterium tuberculosis | 53 |
| | Mycoplasma PG50 | 54 |
| | Neisseria meningitidis | 55 |
| | Staphylococcus aureus | 56 |
| | Streptococcus pneumoniae | 57 |
| Fungi | Cryptococcus | 58 |
| Protozoa | Acanthamoeba sp | 59 |
| | Naegleria jamiesoni | 60 |
| | Toxoplasma gondii | 61 |

The probe sequences of the SEQ ID NOs. 29 and 30 are the basis for discriminating the LCMV and the LCMV′ pathogens. Further, the probe sequence of the SEQ ID NO. 44 is basis for detecting the Brucella abortus, the Brucella canis, the Brucella melitensis and the Brucella suis pathogens.

During the step of designing the probe sequence (step 100), the design of the primer sets that correspond to these probe sequences, which are used to amplify the specific DNA sequence of the patient's sample, are also conducted. These primer sets include at least 38 primer sets, which are formed with the DNA sequences assigned to the SEQ ID NOs. 62-134. Similarly, the design of these primer sets is reliably based on the gene sequences of the pathogens of meningitis obtained from a gene bank.

Each primer set includes a 5′ to 3′ forward primer and a 3′ to 5′ reverse primer for amplifying the specific DNA fragments of the pathogens of meningitis. The following Table 2 lists the meningitis pathogens and the SEQ ID NOs. of the corresponding primer sets.

TABLE 2

| | | SEQ ID NO. | |
|---|---|---|---|
| | | of the Prime Set | |
| | | Forward | Reverse |
| Pathogens | | Primer | Primer |
| Virus | hhv1 \grave{ } hhv2 | 62 | 63 |
| | hhv3 | 64 | 65 |
| | hhv4 | 66 | 67 |
| | hhv5 | 68 | 69 |
| | hhv6 \grave{ } hhv7 | 70 | 71 |
| | coxsackievirus a9 \grave{ } | 72 \grave{ } 73 | 74 |
| | coxsackievirus b4 \grave{ } | | |
| | coxsackievirus b5 \grave{ } enterovirus71 | | |
| | echovirus22 | 75 | 76 |
| | Poliovirus | 77 | 78 |
| | la cross | 79 | 80 |
| | St. louis | 81 | 82 |
| | venezuelan equine | 83 | 84 |
| | west nile | 85 | 86 |
| | western equine \grave{ } eastern equine \grave{ } | 87 \grave{ } 88 | 89 |
| | venezuelan equine | | |
| | Japanese B | 90 | 91 |
| | central european | 92 | 93 |
| | russian | 94 | 95 |
| | Powassan | 96 | 97 |
| | Dengue | 98 | 99 |
| | Mumps | 100 | 101 |
| | Measles | 102 | 103 |
| | Adenovirus | 104 | 105 |
| | LCMV | 106 | 117 |
| | HIV-1 | 108 | 109 |
| | hiv-2 | 110 | 111 |
| | inluenza-a | 112 | 113 |
| | inluenza-b | 114 | 115 |
| | htlv-1 | 116 | 117 |
| | htlv-2 | 118 | 119 |
| | Togavirus | 120 | 121 |
| | Rickettsia typhi | 122 | 123 |
| | Rabies virus | 124 | 125 |
| | Parvovirus B19 | 126 | 127 |
| Fungi | Cryptococcus | 128 | 129 |
| Bacteria | | 130 | 131 |
| Protozoa | | 132 \grave{ } 133 | 134 |

The DNA sequences of the SEQ ID NOs. 72-74 constitute two primer sets. In other words, the forward primer of the SEQ ID NOs. 72 and the reverse primer of the SEQ ID NOs. 73 constitute a primer set, while the forward primer of the SEQ ID NOs. 73 and the reverse primer of the SEQ ID NOs. 74 constitute another primer set. Further, these two primer sets are used to amplify the specific DNA fragments of the pathogens coxsackievirus a9, coxsackievirus b4, coxsackievirus b5 and enterovirus 71 in the test sample. Similarly, the DNA sequences of SEQ ID NOs. 87 to 89 also constitute two primer sets, wherein these primer sets are used to amplify the specific DNA fragments of the pathogens westerm equine, eastern equine, Venezuelan equine. Further, the primer sets that are formed with the DNA sequences of the SEQ ID NOs. 132 to 134 are used to amplify the specific DNA fragments of protozoa in the sample.

It is also worth noting that the above primer sets are not limited to be used in the detection chip of the instant invention. These primer sets, besides being used for amplifying the specific DNA fragments of a patient's sample in a detection chip, these primer sets can view as one type of detection kit. As the detection kit is being used, after the specific DNA fragment of the patient's sample is amplified, the product can be placed in an appropriate detection apparatus (not limited to the detection chip), and analysis is conducted to obtain a test result.

During the synthesis of the probes, the probes with the DNA sequences as assigned to SEQ ID NOs. 1-61 are formed (step 102). Further, during the synthetic step (step 102), the 5′ ends of the DNA sequences assigned to the SEQ ID NOs. 1-61 are also modified. Consequently, these probes can covalently bind with the functional groups on the matrix surface to be immobilized on the matrix surface. The modification includes a 5′ amino modification.

Thereafter, the synthesized probes are dissolved in deionized water to form a solution with a concentration of, for example, 200 mmole/L.

A spotting procedure (step 106) is then conducted to spot the probe solutions on the matrix. Depending on the number of spots required, the radius of the spots is about of 50 to 100 microns. Further, depending on the situation, each type of probe solutions can be spotted more than once. The surface area of the matrix is sufficiently large to accommodate tens to thousands of spots. If the matrix is glass, the probe solution is spotted on the matrix and is mobilized on the matrix through covalent bonding due to the 5′ amino modification to the DNA sequences in the previous probe synthesis procedure (step 102).

Thereafter, an incubation step is conducted to maintain the matrix in a moist and humid environment (step 108), wherein the incubation process is conducted at 37 degrees Celsius for three days continuously.

An oven-drying step is performed to oven-dry the matrix (step 110), wherein this oven-drying step is conducted at 80 degrees Celsius for 2 hours.

A matrix cleaning step is then conducted to clean the matrix (step 112), wherein the matrix cleaning step includes performing a cleaning process and a drying process. The cleaning solution used in the cleaning process is formed with a probe buffer solution and deionized water. The probe buffer solution is formed with 1×SSC and 0.1% of sodium dodecyl sulfate (SDS), while SSC is a solution with a pH of about 7 and is formed with 3M of NaCl and 0.3M of sodium citrate. The drying process is accomplished by blowing drying the matrix using nitrogen gas.

A blocking step is then conducted using a blocking solution to block the matrix surface that has not been blocked (step 114). The blocking solution used is, for example, a pH 7 solution formed with 1% bovine serum albumin (BSA) and 0.01 mol/L of phosphate buffer (PB).

A matrix cleaning step (step 116) is again conducted to clean the matrix. This matrix cleaning step includes performing a cleaning process, followed by a drying process. Further, this matrix cleaning step can be repeated for several times until the matrix is completely cleaned. The cleaning solution used in this cleaning step includes, for example, using deionized water to clean the excess blocking solution. The drying process is, for example, using nitrogen gas to blow dry the matrix. In one preferred embodiment, the matrix cleaning step is repeated, for example, for three times.

The fabrication of the detection chip is completed with the aforementioned method. The detection chip comprises a plurality of DNA sequences specific to meningitis. Therefore, the various pathogens of meningitis can be detected concurrently.

It is also worth noting that during the design of the probes (step 100), a plurality of quality control probe sequences can also design. These quality control probes are immobilized on the matrix after the synthesis step and the spotting step, etc. are performed (steps 102 to 116). The quality control probes are related to the sequence of a specific substance in the test sample, which are used to ensure the sample extracted is an effective sample to prevent misjudgment of the test result.

Further, using the above method, the detection chip obtained includes a plurality of probes immobilized on the matrix. Further, each probe is selected from the group of the DNA sequences assigned to the SEQ ID NOs. 1-61. Further, each probe is formed with 15 to 30 deoxyribonucleic acids. The material of the matrix is, for example, glass.

In another embodiment, each DNA sequence assigned to the SEQ ID NOs. 1-61 is immobilized on the matrix, wherein these DNA sequences are used to detect the pathogens of meningitis. Further, the matrix is not only disposed with these 61 probes. Depending on the situation required, the sequences recited in SEQ ID NOs. 1-61 can be repeated for several times to constitute a microarray detection chip with tens or several thousands probes.

Since these probes are related to the various pathogens of meningitis, they can be used to determine whether the patient has contracted meningitis and the type of meningitis.

The method for detecting whether a patient has contracted meningitis using the microarray detection chip, fabricated according to the above method, is detailed in the following.

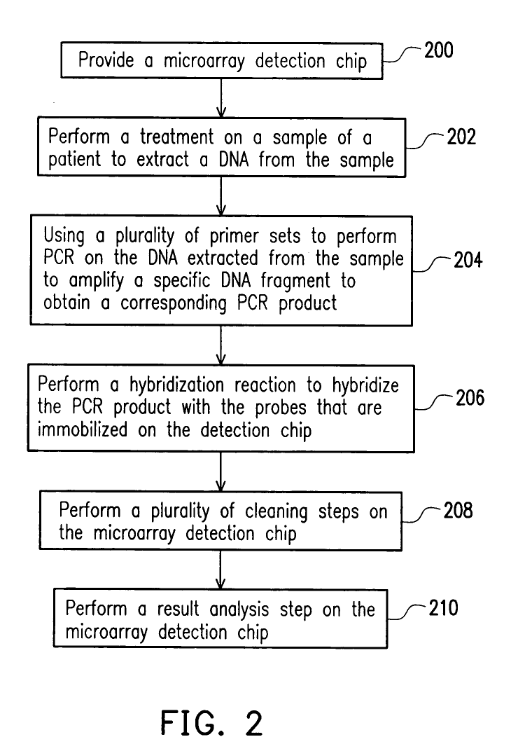

FIG. 2 is a flow diagram illustrating a method for detecting meningitis according to one embodiment of the present invention.

Referring to FIG. 2, a microarray detection chip is provided (step 200), wherein this microarray detection chip is fabricated using, for example, the aforementioned method. Further, this microarray detection chip includes a plurality of probes specific for detecting the pathogens of meningitis. In one embodiment of the invention, besides these specific probes for detecting the pathogens of meningitis, this microarray detection chip further includes quality control probes immobilized thereon.

Thereafter, the sample from a patient is treated to extract the DNA from the sample (step 202), wherein the sample from the patient is, for example, cerebrospinal fluid. The cerebrospinal fluid is treated by placing in a 100 degrees Celsius water bath for about 10 minutes, followed by cooling to 0 degrees Celsius. After spinning at 13000G for 5 minutes, the supernatant is collected and is digested using lysozyme. Chloroform extraction is then conducted on the clear supernatant, followed by precipitating with ethanol to extract the DNA. If the DNA needs to be stored for a longer period time, it can be preserved at −20 degrees Celsius.

A PCR amplification is then conducted on the sample using a plurality of primer sets to amplify specific segments of the DNA to obtain the corresponding PCR product (step 204). The primer sets used in the PCR amplification is selected from the 38 primer sets that are formed with the DNA sequences assigned to the SEQ ID NOs. 62-134.

The reagent used in the PCR amplification includes at least the DNA from the sample, DNA polymerase, the above primer sets and deoxyribonucleoside triphsphate (dNTP), wherein the DNA polymerase is, for example, Tag enzyme. Further, the PCR product is labeled with a PCR product label. To label the PCR product includes, for example, using a labeled primer set, a labeled deoxyuridine triphsphate (dUTP) or a labeled dNTP and the above reagent to perform the PCR amplification. These labels are, for example, Cys, Cy5 or other appropriate fluorescent materials. The PCR product label serves as a reference in the subsequent analysis process for determining whether the PCR product has reacted with the probe.

In the above PCR procedure, symmetric or asymmetric multiplex polymerase chain reaction is conducted. In other words, the numbers of the forward primers and the reverse primers can be different to produce DNA with a single stranded structure in the PCR product. The single stranded DNA is subsequently hybridized with the single stranded probe on the microarray detection chip. If the numbers of the forward primer sets and the reverse primer sets are the same, a thermal denaturation procedure is conducted before the hybridization process to separate the two complementary single DNA strands before proceeding with the hybridization procedure.

The conditions for PCR amplification are shown as follow in Table 3.

| | Reaction | Duration |
|---|---|---|
| STEP | Temperature | Period (min.) |
| 1 | 94 | 5 |
| 2 | 94 | 0.5 |
| 3 | 56 | 0.5 |
| 4 | 72 | 1 |
| 5 | 72 | 5 |

In this embodiment, the PCR amplification is conducted according to the conditions in step 1, followed by repeating the conditions in steps 2 to 4 for 30 cycles, and is concluded according to the conditions in step 5.

Thereafter, a hybridization procedure is conducted to react the PCR product with the probes on microarray detection chip (step 206). The hybridization reaction is conducted, for example, in an environment of about 60 degrees Celsius for about 2 hours. Further, the hybridization reaction includes, for example, using a hybridization buffer, wherein the amount of the hybridization buffer used is same as the amount of the PCR product. The hybridization buffer is formed with 10×SSC and 0.1% SDS. In this procedure, if the sequence of the PCR product with the single stranded structure is complementary to the sequence of the probe, the PCR product is hybridized with the probe. Further, since the PCR product comprises a label, the type of probe that is hybridized can be detected according to this label in a subsequent process.

A plurality of cleaning steps is conducted to clean the microarray detection chip (step 208), wherein the cleaning solution used in these cleaning steps is, for example deionized water. In one preferred embodiment, the microarray detection chip is repeatedly cleaned for three times. Further, with these cleaning procedures (step 208), the PCR product that has not been hybridized with the probe is washed off, leaving only the PCR product that is complementary to the probe.

Thereafter, a result analysis step is performed on the microarray detection chip (step 210). The result analysis step includes performing a scanning procedure and a data analysis procedure, wherein the scanning procedure includes, for example, using a scanner to scan the mircroarray detection chip to obtain information on various test results. The scanner used is provided by, for example, Genomic Solutions. Further, the data analysis procedure includes, for example, using an analysis software that is compatible with the scanner to output the analysis results of the microarray detection chip. Since the PCR product after hybridization is labeled, the scanner can identify whether a label is present at the position of each probe (for example, an emission of a fluorescent signal) during the scanning step with the scanner. Therefore, based on the data analysis procedure, the type of meningitis contracted by the patient is identified. If the microarray detection chip does not include any label, it is an indication that the patient has meningitis.

In accordance to the present invention, the fabrication of a meningitis detection chip is provided, wherein this detection chip includes a plurality of deoxyribonucleotide sequences specific to the various types of meningitis. Therefore, the detection chip is applicable to determine whether the patient has contracted meningitis and the type of meningitis.

The present invention also makes use of the microarray detection chip to attain a large amount and accurate analysis results.

Further, these primer sets (SEQ ID NOs. 62-134) designed according to the present invention are specific to the pathogens of meningitis. Therefore, by using these primer sets to amplify the specific DNA fragments of the patient's sample and the subsequent testing method, whether the patient has meningitis and the type of meningitis are identified.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims and their equivalents.

1. A fabrication method of a meningitis detection chip, the method comprising:
designing a plurality of probe sequences, wherein these probe sequences comprise at lease deoxyribonucleotide sequences assigned to SEQ ID NOs. (Sequence Identifier Number) 1 to 61;
performing a probe synthesis step to synthesize a plurality of probes of the deoxyribonucleotide sequences assigned to the SEQ ID NOs. (Sequence Identifier Number) 1 to 61; and
performing a spotting step to respectively spot the probes on a matrix.

2. The fabrication method of claim 1, wherein the designing of the probe sequences further comprises designing a plurality of primer sets corresponding to the probe sequences to amplify a specific DNA fragment of a sample of a patient, wherein these primer sets comprise 38 primer sets that are formed with DNA sequences assigned to SEQ ID NOs. 62 to 134.

3. The fabrication method of claim 1, wherein the probe synthesis procedure further comprises conducting a 5′ end amino modification of the DNA sequences assigned to the SEQ ID NOs. 1 to 61.

4. The fabrication method of claim 1, subsequent to the probe synthesis step, the method further comprises respectively dissolving the probes in deionized water to form a plurality of probe solutions.

5. The fabrication method of claim 1, wherein subsequent to the spotting procedure, the method further comprises performing an incubation procedure to maintain the matrix in a humid environment.

6. The fabrication method of claim 5, wherein subsequent to the incubation step, the method further comprises:
performing an oven-drying step to dry the matrix; and
performing a matrix cleaning step to clean the matrix.

7. The method of claim 6, wherein the matrix cleaning step further comprises:
performing a cleaning process to clean the matrix; and
performing a drying process to dry the matrix.

8. The method of claim 6, wherein subsequent to the matrix cleaning step, the method further comprises:
performing a blocking step using a blocking solution to block a matrix surface that is not spotted; and
performing another matrix cleaning step to clean the matrix.

9. The method of claim 1, wherein a spot formed by the spotting procedure has a radius of about 50 to 300 microns.

10. A microarray detection chip applicable for detecting whether a patient has contracted meningitis, the microarray detection chip comprising:
a plurality of probes immobilized on a matrix, and these probes are selected from the group of deoxyribonucleotide sequences assigned to SEQ ID NOs. (Sequence Identifier Number) 1 to 61.

11. The detection chip of claim 10, wherein the matrix is immobilized with each of the deoxyribonucleotide sequences assigned to the SEQ ID NOs. 1 to 61.

12. The detection chip of claim 10 further comprising a plurality of quality control probes immobilized on the matrix.

13. A meningitis detection method applicable for used in detecting whether a patient has meningitis, the method comprising:
providing a microarray detection chip of claim 10;
treating a sample of the patient to extract a deoxyribonucleic acid (DNA) from the sample;
using a plurality of primer sets to perform a polymerase chain reaction (PCR) on the DNA to amplify a specific fragment of the DNA to obtain a corresponding PCR product, wherein the primer sets used in the PCR are selected from the group consisting of 38 primer sets, which are formed with DNA sequences assigned to SEQ ID NOs. 62 to 134;
performing a hybridization reaction to react the PCR product with the probes on the microarray detection chip; and
performing a result analysis on the microarray detection chip.

14. The detection method of claim 13, wherein the PCR product is labeled with a label.

15. The detection method of claim 14, wherein the label comprises a fluorescent material.

16. The detection method of claim 14, wherein the labeling of the PCR product comprises using a primer with the label, deoxyuridine triphosphate (dUTP) with the label, and dexoyribonucleoside triphosphate (dNTP) with the label to perform the PCR.

17. The detection method of claim 13, wherein subsequent to the hybridization reaction, a plurality of cleaning steps is performed to clean the microarray detection chip.

18. The detection method of claim 13, wherein the microarray detection chip further comprises immobilizing a plurality of quality control detection chips thereon.

19. The detection method of claim 13 further comprising:
performing a scanning process on the microarray detection chip to attain a plurality of test information; and
performing a data analysis process on the test information.

20. The detection method of claim 13, wherein the sample comprises a cerebrospinal fluid.

21. A primer set for use on meningitis detection, the primer set is selected from the group of 38 primer sets that are formed with deoxyribnucleotide sequences assigned to SEQ ID NOs. 62 to 134.

22. The primer set of claim 21, wherein the primer set is a detection kit, and when the detection kit is used to amplify a specific DNA fragment of a sample of a patient, a resulting product is placed inside a detection device for analysis.
